(19) United States
(12) Patent Application Publication
(10) Pub. No.: US 2022/0354491 A1
(43) Pub. Date: Nov. 10, 2022

(54) SURGICAL STAPLING DEVICE WITH WRISTED END EFFECTOR

(71) Applicant: Covidien LP, Mansfield, MA (US)

(72) Inventor: Jacob C. Baril, Norwalk, CT (US)

(21) Appl. No.: 17/308,156

(22) Filed: May 5, 2021

(51) Int. Cl. A61B 17/068; A61B 17/072

(52) U.S. Cl. CPC A61B 17/0686; A61B 17/072; A61B 2017/07257; A61B 2017/07271; A61B 2017/07278; A61B 2017/07285

(57) ABSTRACT

A surgical stapling device includes a tool assembly and an adapter assembly. The tool assembly is coupled to the adapter assembly to facilitate articulation of the tool assembly in relation to the adapter assembly about different axes of articulation. The tool assembly includes a drive assembly having a drive member that can bend in any direction about the different articulation axes to allow actuation of the stapling device when the tool assembly is in an articulated position.

### FIELD

This disclosure is directed to surgical stapling devices, and more particularly, to surgical stapling devices with wristed end effectors.

### BACKGROUND

Various types of surgical devices used to endoscopically treat tissue are known in the art, and are commonly used, for example, for closure of tissue or organs in transection, resection, and anastomoses procedures, for occlusion of organs in thoracic and abdominal procedures, and for electrosurgically fusing or sealing tissue.

One example of such a surgical device is a surgical stapling device. Typically, surgical stapling devices include tool assembly having an anvil assembly and a cartridge assembly, and a drive assembly. Typically, the drive assembly includes a flexible drive beam and a clamp member that is supported on a distal end of the drive beam. The drive assembly is movable to advance the clamp member through the tool assembly to approximate the cartridge and anvil assemblies and to advance an actuation sled through the cartridge assembly to eject staples from the cartridge assembly.

During laparoscopic or endoscopic surgical procedures, access to a surgical site is achieved through a small incision or through a narrow cannula inserted through a small entrance wounds in a patient. Because of limited area available to access the surgical site, many endoscopic devices include mechanisms for articulating the tool assembly of the device in relation to a body portion of the device to improve access to tissue to be treated. Due to the configuration of the drive beam, articulation of the tool assembly is limited to a single plane.

A continuing need exists in the art for a surgical device that can articulate in different planes.

### SUMMARY

This application is directed to a surgical stapling device that includes a tool assembly and an adapter assembly. The tool assembly is coupled to the adapter assembly to facilitate articulation of the tool assembly in relation to the adapter assembly about different axes of articulation. The tool assembly includes a drive assembly having a drive member that can bend in any direction about the different articulation axes to allow actuation of the stapling device when the tool assembly is in an articulated position.

One aspect of this disclosure is directed to a tool assembly including a first jaw, a second jaw, and a drive assembly. The first jaw is pivotably coupled to the second jaw to facilitate movement of the tool assembly between open and clamped positions. The drive assembly includes a drive member having a circular cross-section and a working end. The drive member includes a proximal portion and a distal portion and is bendable in any direction. The working end is coupled to the distal portion of the drive member and is engaged with the first and second jaws such that the drive assembly is movable between advanced and retracted positions to move the tool assembly between the open and clamped positions.

In aspects of the disclosure, the drive member is formed from nitinol.

In some aspects of the disclosure, the first jaw includes an anvil, and the second jaw includes a cartridge assembly.

In certain aspects of the disclosure, the cartridge assembly includes a staple cartridge that includes a cartridge body, staples, pushers, and an actuation sled.

In aspects of the disclosure, the cartridge body defines staple receiving slots and a central knife slot, and the staple receiving slots are positioned on opposite sides of the knife receiving slot.

In some aspects of the disclosure, the working end of the drive assembly has an I-beam configuration and includes a first beam, a second beam, and a strut interconnecting the first and second beams.

In certain aspects of the disclosure, the vertical strut includes a cutting blade.

In aspects of the disclosure, the working end of the drive assembly is secured to the distal portion of the drive member by welding.

In some aspects of the disclosure, the working end of the drive assembly is secured to the distal portion of the drive member using a pin.

Other aspects of the disclosure are directed to a surgical device including an adapter assembly and a tool assembly. The adapter assembly has a proximal portion and a distal portion and includes articulation links. The tool assembly is pivotably coupled to the distal portion of the adapter assembly and includes a first jaw, a second jaw, and a drive assembly. The first jaw is pivotably coupled to the second jaw to facilitate movement of the tool assembly between open and clamped positions. The drive assembly includes a drive member having a circular cross-section and a working end. The drive member includes a proximal portion and a distal portion and is bendable in any direction. The working end is coupled to the distal portion of the drive member and is engaged with the first and second jaws such that the drive assembly is movable between advanced and retracted positions to move the tool assembly between the open and clamped positions.

In certain aspects of the disclosure, the tool assembly is coupled to the distal portion of the adapter assembly with a gimbal that facilitates articulation of the tool assembly in relation to the adapter assembly about different articulation axes.

In aspects of the disclosure, the articulation links are coupled to the gimbal and movable to articulate the tool assembly about the different articulation axes.

In aspects of the disclosure, the surgical device includes a handle assembly, and the proximal portion of the adapter assembly is coupled to the handle assembly.

Other aspects of the disclosure are directed to a surgical stapling device that includes an adapter assembly and a tool assembly. The adapter assembly has a proximal portion and a distal portion and includes articulation links. The tool assembly is pivotably coupled to the distal portion of the adapter assembly and includes an anvil, a cartridge assembly, and a drive assembly. The cartridge assembly is pivotably coupled to the anvil to facilitate movement of the tool assembly between open and clamped positions and includes a staple cartridge and a channel member. The staple cartridge includes a cartridge body, staples, pushers, and an actuation sled. The cartridge body defines staple receiving slots and a central knife slot. The staple receiving slots are positioned on opposite sides of the knife receiving slot and receive the staples and the pushers. The drive assembly includes a drive member having a circular cross-section, and a working end. The drive member includes a proximal portion and a distal portion and is bendable in any direction. The working end is coupled to the distal portion of the drive member and is engaged with the anvil and the cartridge assembly such that the drive assembly is movable between advanced and retracted positions to move the tool assembly between the open and clamped positions and to eject the staples from the cartridge body.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of a surgical stapling device according to aspects of this disclosure with a tool assembly in a non-articulated, open position;

FIG. 2 is an exploded, side perspective view of a tool assembly of the surgical stapling device of FIG. 1;

FIG. 3 is an enlarged view of the indicated area of detail shown in FIG. 1;

FIG. 4 is a side perspective view of a distal portion of a drive assembly of the surgical stapling device shown in FIG. 1;

FIG. 5 is a side perspective view of the distal portion of the surgical stapling device shown in FIG. 1 showing the distal portion of the drive assembly shown in phantom and the tool assembly in an articulated position; and

FIG. 6 is a side perspective view of the distal portion of the drive assembly shown in FIG. 5 in an articulated position.

### DETAILED DESCRIPTION

The disclosed surgical stapling device will now be described in detail with reference to the drawings in which like reference numerals designate identical or corresponding elements in each of the several views. However, it is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

As used herein, the term “distal” refers to the portion of the stapling device that is being described which is further from a user during use of the device in its customary manner, while the term “proximal” refers to the portion of the stapling device that is being described which is closer to a user during use of the device in its customary manner. In addition, directional terms such as front, rear, upper, lower, top, bottom, and similar terms are used to assist in understanding the description and are not intended to limit the disclosure. As used herein, the terms “parallel”, “perpendicular”, and “aligned” are understood to include relative configurations that are substantially parallel, substantially perpendicular, and substantially aligned, i.e., up to about + or −10 degrees from true parallel, true perpendicular, true alignment.

This application is directed to a surgical stapling device that includes a tool assembly and an adapter assembly. The tool assembly is coupled to the adapter assembly to facilitate articulation of the tool assembly in relation to the adapter assembly about different axes of articulation. The tool assembly includes a drive assembly having a drive member that can bend in any direction about the different articulation axes to allow actuation of the stapling device when the tool assembly is in an articulated position.

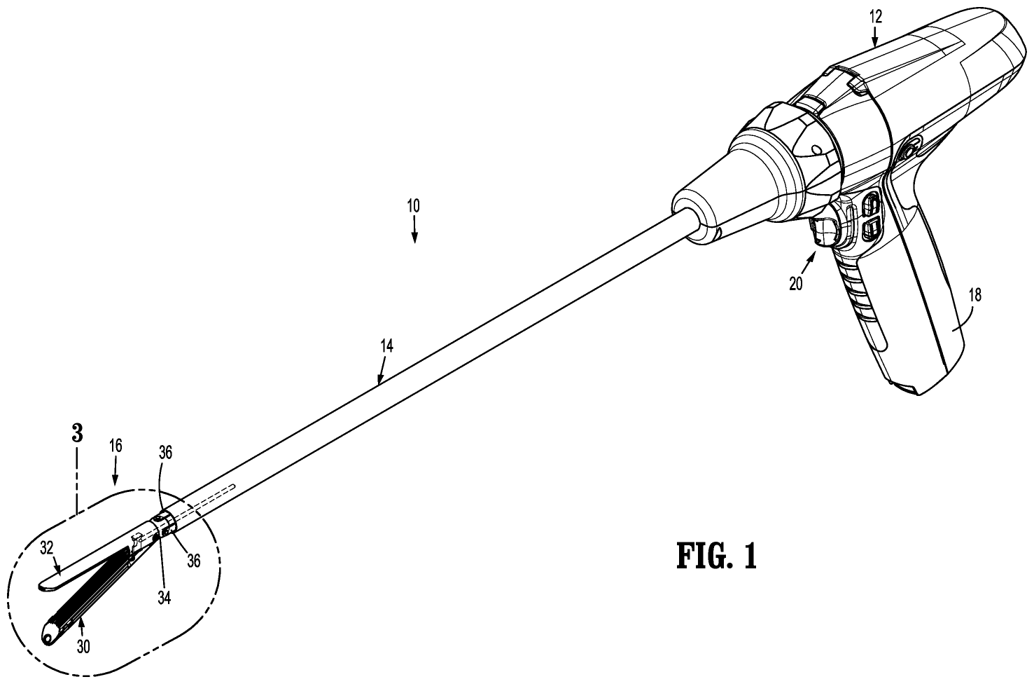

FIG. 1 illustrates a surgical stapling device shown generally as stapling device 10 that includes a handle assembly 12, an elongate body or adapter assembly 14, and a tool assembly 16. As illustrated, the handle assembly 12 is powered and includes a stationary handgrip 18 and actuation buttons 20. The actuation buttons 20 are operable to actuate various functions of the tool assembly 16 via the adapter assembly 14 including approximation, stapling, cutting, and articulation. In certain aspects of the disclosure, the handle assembly 12 supports batteries (not shown) that provide power to the handle assembly 12 to operate the stapling device 10. Although the stapling device 10 is illustrated as a powered stapling device, it is envisioned that the features of this disclosure are suitable for use with manually powered surgical stapling devices as well as robotically controlled stapling devices. It is also envisioned that aspects of this disclosure are suitable for use with surgical devices other than stapling devices that include tool assemblies that articulate in relation to a body portion of the surgical device.

The tool assembly 16 of the stapling device 10 includes a cartridge assembly 30 and an anvil 32 that are pivotably coupled together by screws or pins 33. The anvil 32 defines openings 32a that receive the screws 33. The cartridge assembly 30 and the anvil 32 are coupled together such that the tool assembly 16 can pivot between open and clamped positions. In aspects of the disclosure, the tool assembly 16 is coupled to a distal portion of the adapter assembly 14 by a multi-axis gimbal joint 34. The adapter assembly 14 includes four articulation links 36 (only two are shown) that can be moved longitudinally via operation of the actuation buttons 20 to articulate the tool assembly 16 in relation to the adapter assembly 14 about different articulation axes from a non-articulated position to articulated positions. In the non-articulated position, the longitudinal axis of the tool assembly 16 is coaxial with a longitudinal axis of the adapter assembly 14. In the articulated positions, the longitudinal axis of the tool assembly 16 and the longitudinal axis of the adapter assembly 14 define acute angles.

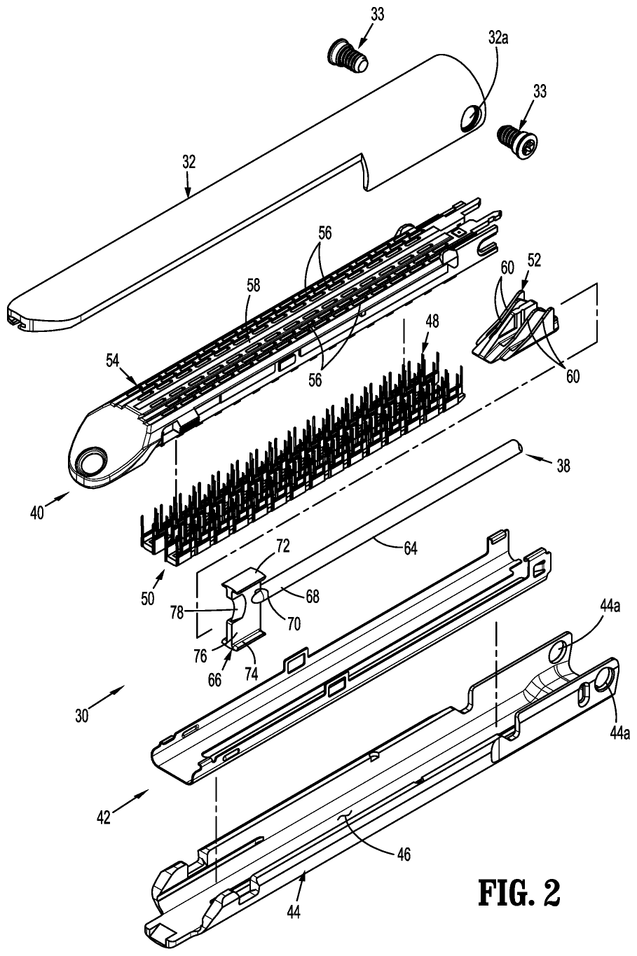

FIG. 2 illustrates the tool assembly 16 which includes a first jaw supporting the cartridge assembly 30, a second jaw supporting the anvil 32, and a drive assembly 38. The cartridge assembly 30 includes a staple cartridge 40, a staple guard 42, and a channel member 44. The channel member 44 defines a cavity 46 that receives the staple cartridge 40 and openings 44a that receive the screws 33. In aspects of the disclosure, the staple cartridge 40 can be releasable from the cavity 46 of the channel member 44 to facilitate replacement of the staple cartridge 40 from the channel member 44 and reuse the stapling device 10. Alternately, the staple cartridge 40 can be fixedly received within the cavity 46 of the channel member 44.

The staple cartridge 40 includes staples 48, pushers 50, an actuation sled 52, and a cartridge body 54. The cartridge body 54 defines a plurality of staple receiving slots 56 and a central knife slot 58. In aspects of the disclosure, the staple receiving slots 56 are aligned in rows on opposite sides of the central knife slot 58 although other arrays of staple receiving slots 56 are envisioned. Each of the staple receiving slots 56 receives one of the staples 48 and a pusher 50. The actuation sled 52 includes ramps 60 that are movable through the cartridge body 54 into engagement with the pushers 50 to eject the staples 48 from the cartridge body 54. The staple guard 42 is secured to a bottom of the cartridge body 54 to retain the staples 48 and the pushers 50 within the cartridge body 54. For a more detailed description of the construction and operation of an exemplary cartridge assembly, see U.S. Pat. No. 7,891,534.

FIGS. 2-4 illustrate the drive assembly 38 of the stapling device 10 (FIG. 1) which includes a drive member 64 and a working end 66. In aspects of the disclosure, the drive member 64 includes a proximal portion (not shown) and a distal portion 68. The proximal portion is coupled to a drive mechanism (not shown) within the adapter assembly 14 of the stapling device 10 (FIG. 1) such that longitudinal movement of the drive mechanism causes longitudinal movement of the drive assembly 38. The distal portion 68 of the drive member 64 is secured to the working end 66 of the drive assembly 38 such that longitudinal movement of the drive member 64 causes longitudinal movement of the working end 66. In aspects of the disclosure, the working end 66 of the drive assembly 38 is welded or pinned to the distal portion 68 of the drive member 64. In some aspects of the disclosure, the distal portion 68 of the drive member 64 defines a slot 70 that receives a portion of the working end 66 of the drive assembly 38.

In aspects of the disclosure, the working end 66 of the drive assembly 38 is rigid and has an I-beam configuration and includes a first beam 72, a second beam 74, and a vertical strut 76. The first beam 72 is supported on one end of the vertical strut 76 and the second beam 74 is supported on an opposite end of the vertical strut 76. In aspects of the disclosure, the vertical strut 76 includes or supports a cutting blade 78. The first beam 72 is engaged with the anvil 52 and the second beam 74 is engaged with the channel member 44 such that longitudinal movement of the working end 66 of the drive assembly 38 causes the tool assembly 16 to move between the open and clamped positions. The vertical strut 76 of the working end 68 of the drive assembly 38 is positioned proximally of and in alignment with the actuation sled 52 such that advancement of the working end 68 of the drive assembly 38 in relation to the anvil 32 and the cartridge assembly 30 causes advancement of the actuation sled 52 within the cartridge assembly 30 to eject the staples from the cartridge body 54. In aspects of the disclosure, the working end 68 of the drive assembly 38 is formed from metal, e.g., stainless steel, although other materials of construction are envisioned.

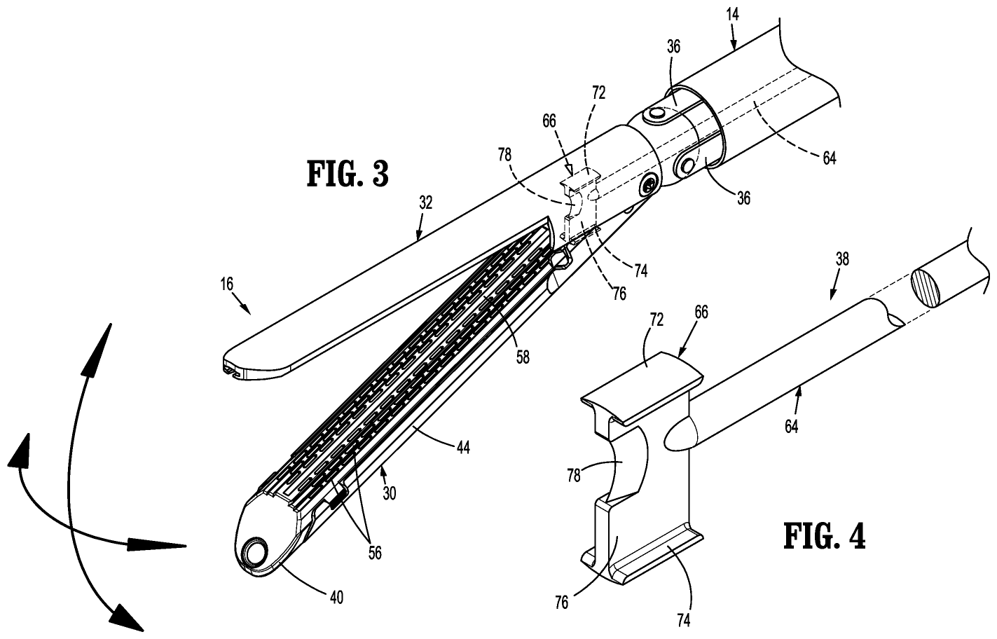

The drive member 64 has a circular cross-section and is formed of a flexible material that has a high compressive strength that can withstand buckling during approximation and firing of the stapling device 10 (FIG. 1). In aspects of the disclosure, the drive member 64 is formed from nitinol although other materials of construction are envisioned. When the tool assembly 16 is articulated in relation to the adapter assembly 14 in one or more of the directions indicated by arrows A and B in FIG. 3, the drive member 64 can bend about the axis or axes of articulation.

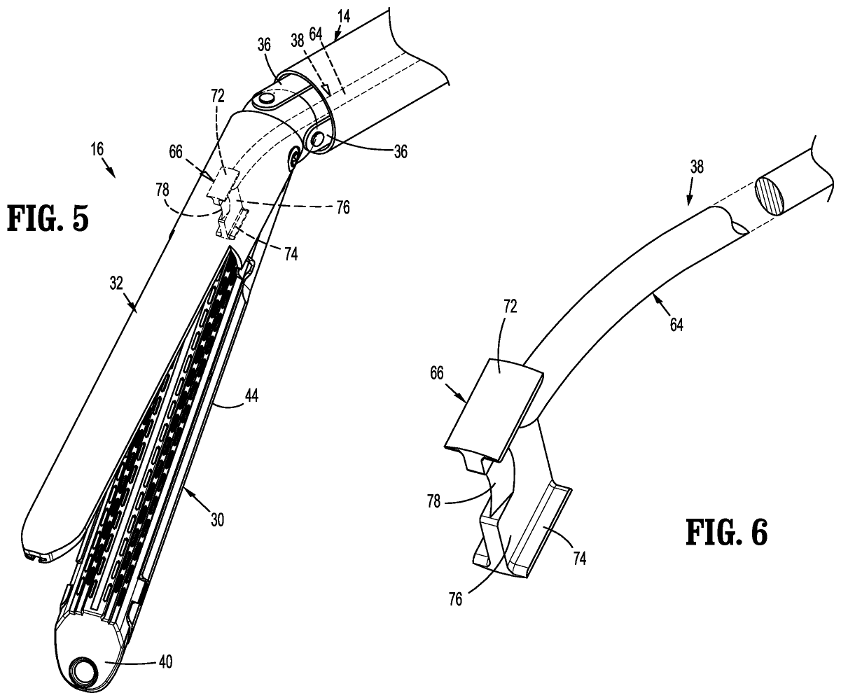

FIGS. 5 and 6 illustrate the tool assembly 16 in an articulated position. As shown, when the tool assembly 16 is articulated in relation to the adapter assembly 14, the drive member 64 of the drive assembly 38 bends about the axis of articulation. Because of the circular configuration of the drive member 64, the drive member 64 can bend in any direction to facilitate articulation of the tool assembly 16 about the different articulation axes.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects. Accordingly, the disclosure is not to be limited by what has been particularly shown and described, except as indicated by the appended claims.

What is claimed is:

1. A tool assembly comprising:
a first jaw;
a second jaw pivotably coupled to the first jaw to facilitate movement of the tool assembly between open and clamped positions; and
a drive assembly including a drive member and a working end, the drive member having a circular cross-section and including a proximal portion and a distal portion, the drive member being bendable in any direction, the working end coupled to the distal portion of the drive member and engaged with the first and second jaws such that the drive assembly is movable between advanced and retracted positions to move the tool assembly between the open and clamped positions.

2. The tool assembly of claim 1, wherein the drive member is formed from nitinol.

3. The tool assembly of claim 1, wherein the first jaw includes an anvil, and the second jaw includes a cartridge assembly.

4. The tool assembly of claim 3, wherein the cartridge assembly includes a staple cartridge, the staple cartridge including a cartridge body, staples, pushers, and an actuation sled, the cartridge body defining staple receiving slots and a central knife slot, the staple receiving slots positioned on opposite sides of the knife receiving slot.

5. The tool assembly of claim 4, wherein the working end of the drive assembly has an I-beam configuration and includes a first beam, a second beam, and a strut interconnecting the first and second beams.

6. The tool assembly of claim 5, wherein the vertical strut includes a cutting blade.

7. The tool assembly of claim 6, wherein the working end of the drive assembly is secured to the distal portion of the drive member by welding.

8. The tool assembly of claim 6, wherein the working end of the drive assembly is secured to the distal portion of the drive member using a pin.

9. A surgical device comprising:
an adapter assembly having a proximal portion and a distal portion, the adapter assembly including articulation links; and
a tool assembly pivotably coupled to the distal portion of the adapter assembly, the tool assembly including:
a first jaw;
a second jaw pivotably coupled to the first jaw to facilitate movement of the tool assembly between open and clamped positions; and
a drive assembly including a drive member having a circular cross-section and a working end, the drive member including a proximal portion and a distal portion and being bendable in any direction, the working end coupled to the distal portion of the drive member and engaged with the first and second jaws such that the drive assembly is movable between advanced and retracted positions to move the tool assembly between the open and clamped positions.

10. The surgical device of claim 9, wherein the drive member is formed from nitinol.

11. The surgical device of claim 9, wherein the first jaw includes an anvil, and the second jaw includes a cartridge assembly.

12. The surgical device of claim 11, wherein the cartridge assembly includes a staple cartridge, the staple cartridge including a cartridge body, staples, pushers, and an actuation sled, the cartridge body defining staple receiving slots and a central knife slot, the staple receiving slots positioned on opposite sides of the knife receiving slot and receiving the staples.

13. The surgical device of claim 12, wherein the working end of the drive assembly has an I-beam configuration and includes a first beam, a second beam, and a strut interconnecting the first and second beams.

14. The surgical device of claim 13, wherein the vertical strut includes a cutting blade.

15. The surgical device of claim 14, wherein the working end of the drive assembly is secured to the distal portion of the drive member by welding.

16. The surgical device of claim 14, wherein the working end of the drive assembly is secured to the distal portion of the drive member using a pin.

17. The surgical device of claim 9, wherein the tool assembly is coupled to the distal portion of the adapter assembly with a gimbal, the gimbal facilitating articulation of the tool assembly in relation to the adapter assembly about different articulation axes.

18. The surgical device of claim 17, wherein the articulation links are coupled to the gimbal and movable to articulate the tool assembly about the different articulation axes.

19. The surgical device of claim 18, further including a handle assembly, the proximal portion of the adapter assembly coupled to the handle assembly.

20. A surgical stapling device comprising:
an adapter assembly having a proximal portion and a distal portion, the adapter assembly including articulation links; and
a tool assembly pivotably coupled to the distal portion of the adapter assembly, the tool assembly including:
an anvil;
a cartridge assembly pivotably coupled to the anvil to facilitate movement of the tool assembly between open and clamped positions, the cartridge assembly including a channel member and a staple cartridge, the staple cartridge including a cartridge body, staples, pushers, and an actuation sled, the cartridge body defining staple receiving slots and a central knife slot, the staple receiving slots positioned on opposite sides of the knife receiving slot and receiving the staples and the pushers; and
a drive assembly including a drive member having a circular cross-section and a working end, the drive member including a proximal portion and a distal portion and being bendable in any direction, the working end coupled to the distal portion of the drive member and engaged with the anvil and the cartridge assembly such that the drive assembly is movable between advanced and retracted positions to move the tool assembly between the open and clamped positions and to eject the staples from the cartridge body.
